(19) United States
(12) Patent Application Publication
(10) Pub. No.: US 2018/0149644 A1
(43) Pub. Date: May 31, 2018

(54) BIOSENSOR

(71) Applicant: METAL INDUSTRIES RESEARCH AND DEVELOPMENT CENTRE, Kaohsiung (TW)

(72) Inventors: Tung-Chieh YANG, Kaohsiung (TW); Yu-Jie LAN, Kaohsiung (TW); Tsung-Chih YU, Kaohsiung (TW); Min-Hang WENG, Kaohsiung (TW); Siang-Wen LAN, Kaohsiung (TW); Ru-Yuan YANG, Kaohsiung (TW); Tien-Chun TSAI, Kaohsiung (TW); Hsien-Chang CHANG, Kaohsiung (TW)

(21) Appl. No.: 15/395,787

(22) Filed: Dec. 30, 2016

(30) Foreign Application Priority Data
Nov. 25, 2016 (TW) 105138782

(51) Int. Cl. G01N 33/543; H01P 7/08

(52) U.S. Cl. CPC G01N 33/54373; H01P 7/082

(57) ABSTRACT

A biosensor includes a microstrip resonator, a collection actuator disposed adjacent to and spaced from the microstrip resonator, a port unit connected to a network analyzer and coupled with the microstrip resonator, and a substrate. The microstrip resonator includes a collecting portion provided with an analyte-specific reagent (ASR) for binding an analyte in a suspension. The collection actuator and the microstrip resonator are applied with alternating current (AC) electric energy to induce AC electrokinetic stirring in the suspension for actuating the binding. The microstrip resonator resonates at a resonant frequency associated with the binding so that the analyte can be quantified.

### CROSS-REFERENCE TO RELATED APPLICATION

This application claims priority of Taiwanese Patent Application No. 105138782, filed on Nov. 25, 2016.

### FIELD

The disclosure relates to a biosensor, and more particularly to a biosensor for detection of an analyte in a suspension.

### BACKGROUND

Referring to FIG. 1, a principle of detection by affinity is illustrated. An analyte-specific reagent (ASR), which is exemplified by antibody molecules 11, is adsorbed on a surface for binding with an analyte, which is exemplified by antigen molecules 12, in a suspension through affinity between the ASR and the analyte. However, the ASR and the analyte are usually nanoscale in dimensions, resulting in a slow binding therebetween due to influence of Brownian motion and diffusion, and resulting in difficulty of observing a binding result.

### SUMMARY

Therefore, an object of the disclosure is to provide a biosensor configured for detection of an analyte in a suspension when used in combination with a network analyzer that can alleviate at least one of the drawbacks of the prior art.

According to the disclosure, the biosensor includes a substrate, a microstrip resonator, a collection actuator and a port unit. The microstrip resonator is disposed on the substrate, and includes a collecting portion that is provided with an analyte-specific reagent (ASR) thereon for binding the analyte in the suspension through affinity between ASR and the analyte. The collection actuator is disposed on the substrate, and is disposed adjacent to and spaced apart from the microstrip resonator. The collection actuator and the microstrip resonator are applied with alternating current (AC) electric energy so as to induce AC electrokinetic stirring in the suspension for actuating the binding between the ASR and the analyte. The port unit is disposed on the substrate, and is configured to be electrically connected to the network analyzer, and to be electromagnetically coupled with the microstrip resonator such that the microstrip resonator resonates at a resonant frequency which varies based on the binding between the ASR and the analyte, so as to enable the analyte to be quantified according to variation in the resonant frequency observed through the network analyzer.

### BRIEF DESCRIPTION OF THE DRAWINGS

Other features and advantages of the disclosure will become apparent in the following detailed description of the embodiments with reference to the accompanying drawings, of which:

FIG. 1 is schematic diagram illustrating a principle of detection by affinity between an analyte and an analyte-specific reagent (ASR);

FIG. 2 is a schematic top view illustrating a first embodiment of a biosensor according to the disclosure;

FIG. 3 is a schematic side view illustrating one embodiment of a microstrip resonator of the biosensor;

FIG. 4 is a schematic diagram illustrating alternating current electric energy applied between the microstrip resonator and a collection actuator;

FIG. 5 is a schematic diagram for illustrating relationship between resonant frequency and different microstrip resonators;

FIG. 6 is a schematic top view illustrating a second embodiment of the biosensor according to the disclosure;

FIG. 7 is a fragmentary view illustrating the collection actuator, the microstrip resonator and a limiter of the second embodiment of the biosensor;

FIG. 8 is a schematic diagram illustrating the alternating current electric energy applied between the microstrip resonator and the collection actuator so as to induce AC electrokinetic stirring in a suspension; and

FIG. 9 is a schematic top view illustrating a third embodiment of the biosensor according to the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

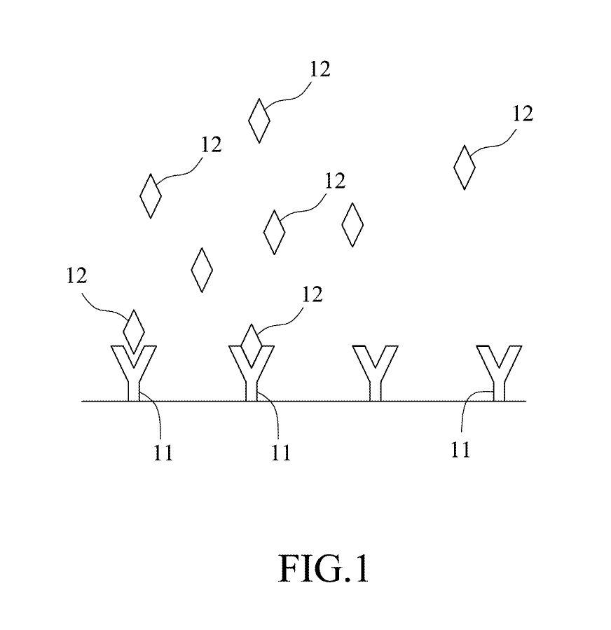

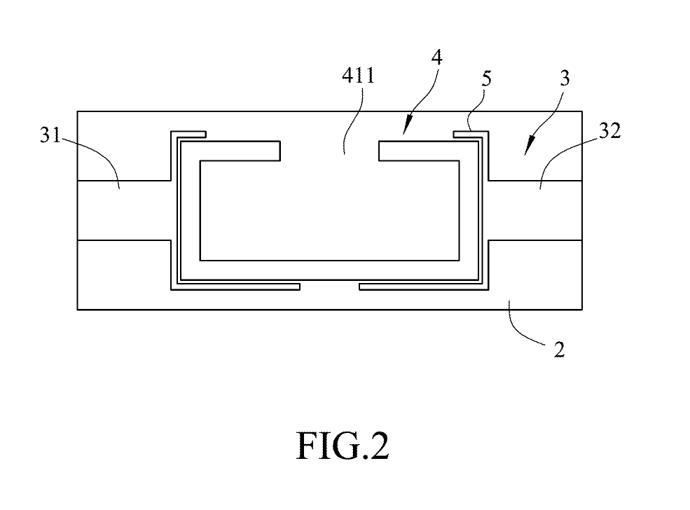

Referring to FIGS. 1 and 2, a first embodiment of a biosensor is illustrated. The biosensor is configured for detection of an analyte 12 in a suspension when used in combination with a network analyzer (not shown). The biosensor includes a substrate 2, a microstrip resonator 4 disposed on the substrate 2, a collection actuator 5 disposed on the substrate 2, and a port unit 3 disposed on the substrate 2. The microstrip resonator 4 is provided with an analyte-specific reagent (ASR) thereon for binding with the analyte in the suspension through affinity between the ASR and the analyte.

It is worth to note that the analyte is exemplified by antigen molecules 12 for illustration in the following, but the implementation of the analyte is not limited thereto. Also, embodiment of the biosensor is not limited to what is illustrated in FIG. 2; shapes and placements of the aforementioned components may vary depending on different considerations in practice.

The port unit 3 is configured to be electrically connected to the network analyzer, and is to be electromagnetically coupled with the microstrip resonator 4 for receiving a test signal from the network analyzer such that the microstrip resonator 4 resonates at a resonant frequency which varies based on the binding between the ASR and the analyte, so as to enable the analyte to be quantified according to variation in the resonant frequency observed through the network analyzer. The port unit 3 includes a first port 31 and a second port 32 via which the port unit 3 is electrically connected to the network analyzer for the latter to detect the resonant frequency of the microstrip resonator 4.

The microstrip resonator 4 is disposed between the first and second ports 31, 32 of the port unit 3. In the first embodiment, the microstrip resonator 4 is a single split-ring resonator formed with a split 411, and is implemented to have a substantially rectangular shape with the split 411. It should be noted that configurations of the microstrip resonator 4 may vary in other embodiments, and are not limited to the disclosure herein. For example, in other embodiments, the microstrip resonator 4 may be an open-end resonator, a stub resonator, and so on.

In the first embodiment, the collection actuator 5 is formed on a portion of the port unit 3 adjacent to the microstrip resonator 4, and is implemented to surround the microstrip resonator 4.

The microstrip resonator 4, the port unit 3 and the collection actuator 5 are made of an electrically conductive material selected from the group consisting of metal, carbon and a combination thereof, and are fabricated by microelectromechanical systems (MEMS) process technology, which may include steps of photolithography, deposition, etching, patterning, and so on. Since MEMS process technology is well known by a person who is skilled in the art, details related to fabrication of the biosensor are omitted herein. It is worth noting that technologies such as screen printing and laser engraving are feasible ways to fabricate the biosensor.

Referring to FIG. 4, in one embodiment, the microstrip resonator 4 includes a collecting portion 419 that is provided with the ASR thereon for binding with the analyte in the suspension through affinity between the ASR and the analyte, and that extends to approach the collection actuator 5. Herein, the ASR is implemented by antibody molecules 11 (see FIG. 1) that are highly specific to antigen molecules 12 (see FIG. 1), which serve as the analyte. In certain embodiments, the ASR may be provided on the collecting portion 419 through specific functional groups that are capable of binding with the ASR. In practice, the specific functional groups are first fixed to a surface of the collecting portion 419 of the microstrip resonator 4, and the ASR is then bound with the specific functional groups so as to fix the ASR on the collecting portion 419. It should be noted that the ASR can be provided on the collection actuator 5 in the same way.

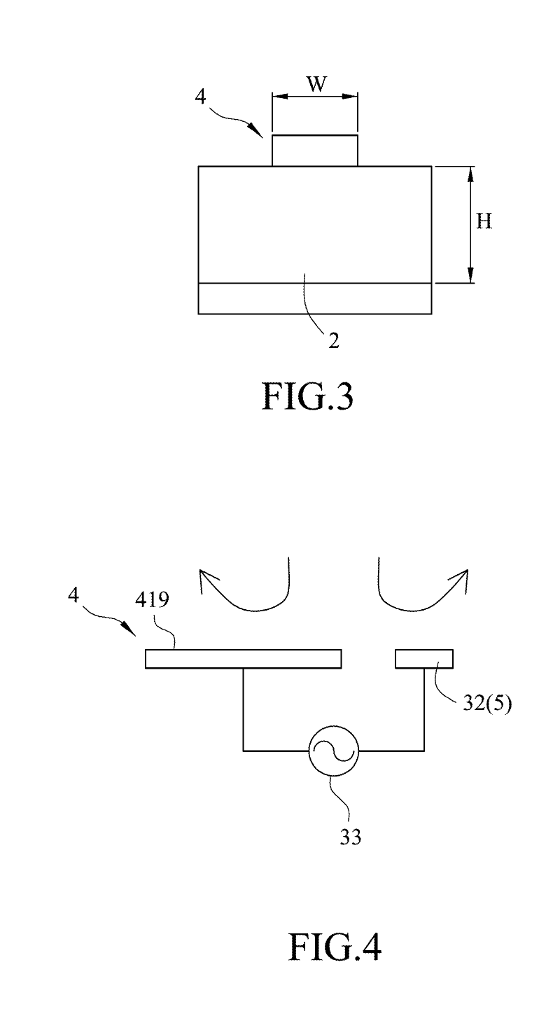

Referring to FIGS. 2 and 3, the resonant frequency at which the microstrip resonator 4 resonates is related to geometries and dimensions of the microstrip resonator 4. In practice, a thickness of the microstrip resonator 4 is substantially in a range between 1 nanometer and 100 micrometers.

When an effective width W of the microstrip resonator 4 is greater than a height H of the substrate 2, an effective dielectric constant ε_{eff} of the microstrip resonator 4 can be approximated by

$${ɛ}_{eff} = \frac{{ɛ}_{r} + 1}{2} + [ \frac{{ɛ}_{r} + 1}{2 \sqrt{1 + 12 ( \frac{H}{W} )}} ] ,$$

where εᵣ is a relative dielectric constant of the substrate 2. In practice, the height H of the substrate 2 is substantially in a range between 0.1 millimeters and 30 millimeters, and the relative dielectric constant εᵣ of the substrate 2 is substantially in a range between 1 and 50. Therefore, the effective dielectric constant ε_{eff} of the microstrip resonator 4 of the biosensor is substantially in a range between 1 and 100.

Similarly, when the effective width W of the microstrip resonator 4 is smaller than the height H of the substrate 2, the effective dielectric constant ε_{eff} of the microstrip resonator 4 can be approximated by

$${ɛ}_{eff} = \frac{{ɛ}_{r} + 1}{2} + \frac{{ɛ}_{r} + 1}{2} [ \frac{1}{\sqrt{1 + 12 ( \frac{H}{W} )}} + 0.04 {( 1 - \frac{H}{W} )}^{2} ] .$$

As a result, in practice, the effective dielectric constant ε_{eff} of the microstrip resonator 4 of the biosensor is substantially in a range between 1 and 10000.

A length L of the microstrip resonator 4 can be approximated by

$$L = \frac{C}{2 f \sqrt{{ɛ}_{eff}}} ,$$

where C is the speed of light, and f is the resonant frequency at which the microstrip resonator 4 resonates. Considering feasibility in practice, the microstrip resonator 4 is designed to resonate at a resonant frequency substantially in a range between 0.3 gigahertz and 100 gigahertz, and the length L of the microstrip resonator 4 is between millimeter scale and centimeter scale, and is substantially in a range between 1 millimeter and 50 centimeters.

Referring to FIGS. 2 and 4, the collection actuator 5 is disposed adjacent to and spaced apart from the microstrip resonator 4. The collection actuator 5 and the microstrip resonator 4 are applied with alternating current (AC) electric energy by an AC power supply 33 so as to induce AC electrokinetic stirring in the suspension for actuating the binding between the ASR and the analyte. In practice, the AC electric energy has a peak-to-peak voltage substantially in a range between 0.01 volts and 30 volts, and a frequency substantially in a range between 0.1 hertz and 100 megahertz. The above-mentioned AC electrokinetic stirring is mainly attributed to AC electro-osmotic flow (ACEOF), dielectrophoresis (DEP) and electro-thermal effect (ETE).

ACEOF is a phenomenon induced by applying AC electrical potential to two parallel electrodes disposed in liquid so as to attract counterions in the liquid to surfaces thereof for forming an electrical double layer. Under influence of tangential electric field, Coulomb force in a direction from a center between the two electrodes toward the two electrodes is formed by the couterions accumulated on the surfaces of the two electrodes. Thereafter, the counterions having moved toward the two electrodes are affected by an alternating magnetic field to form vortices in the liquid.

DEP is a phenomenon in which a force is exerted on a dielectric particle in liquid when the particle is subjected to a non-uniform electric field. When the particle has polarizability higher than that of surrounding medium, the particle will be pushed toward regions of higher electric field, and such phenomenon is called positive DEP. Comparatively, when the particle has polarizability lower than that of the surrounding medium, the particle will be pushed toward regions of lower electric field, and such phenomenon is called negative DEP.

ETE arises from uneven Joule heating due to an electric current flowing through the fluid and henceforward generating heat, so as to cause vortices above the electrodes.

The AC electrokinetic stirring influences motions of the analyte in the suspension and manipulates the same, such that a probability of the binding between the ASR and the analyte is increased. Consequently, in comparison with a situation where the analyte is merely influenced by Brownian motion and diffusion, the AC electrokinetic stirring shortens reaction time of binding between the analyte in the suspension and the ASR. It should be noted that in the first embodiment, the AC electrokinetic stirring is primarily caused by the ACEOF and the DEP.

When the biosensor is being used for detection, at the beginning, the AC electric energy is applied to the microstrip resonator 4 and the collection actuator 5 for a while so as to induce AC electrokinetic stirring in the suspension for actuating the binding between the ASR and the analyte. Then, the AC electric energy is removed, and the network analyzer electrically connected to the first and second ports 31 and 32 inputs therethrough an electromagnetic signal to serve as the test signal so as to detect the resonant frequency of the microstrip resonator 4.

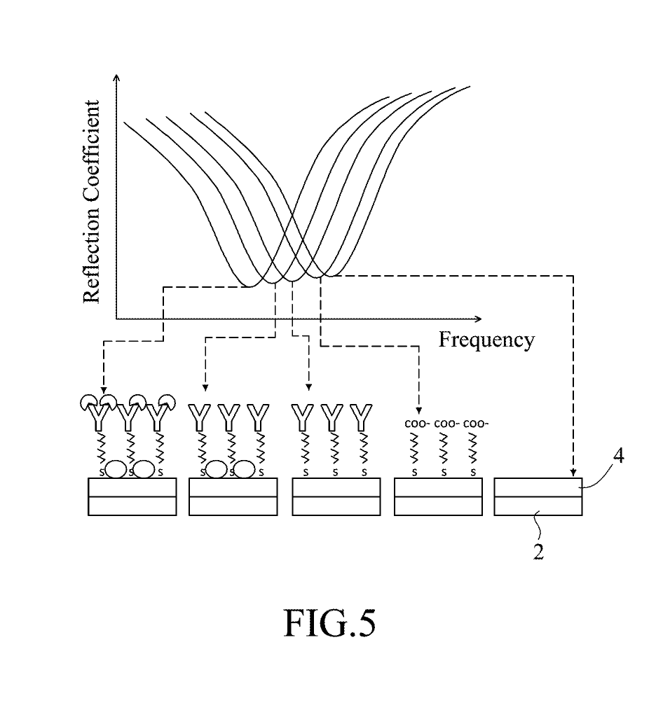

Referring to FIG. 5, given that the length L and the speed of light C are fixed, the resonant frequency f at which the microstrip resonator 4 resonates is inversely proportional to a square root of the effective dielectric constant ε_{eff}, which varies based on the binding between the ASR and the analyte. In FIG. 5, results of detection of relation between reflection coefficient and frequency by the network analyzer are plotted, where the horizontal axis represents frequency, the vertical axis represents reflection coefficient detected by the network analyzer. The five waveforms plotted in FIG. 5 correspond respectively to a microstrip resonator 4 not provided with the ASR, a microstrip resonator 4 provided with the functional groups, a microstrip resonator 4 provided with the ASR that is bound with the functional groups, a microstrip resonator 4 provided with the ASR whose binding with the functional groups is stabilized through blocking, and such microstrip resonator 4 whose ASR has bound with the analyte. Frequency shift can be apparently observed from FIG. 5. Therefore, by observing the variation in the resonant frequency, the analyte in the suspension can be quantified.

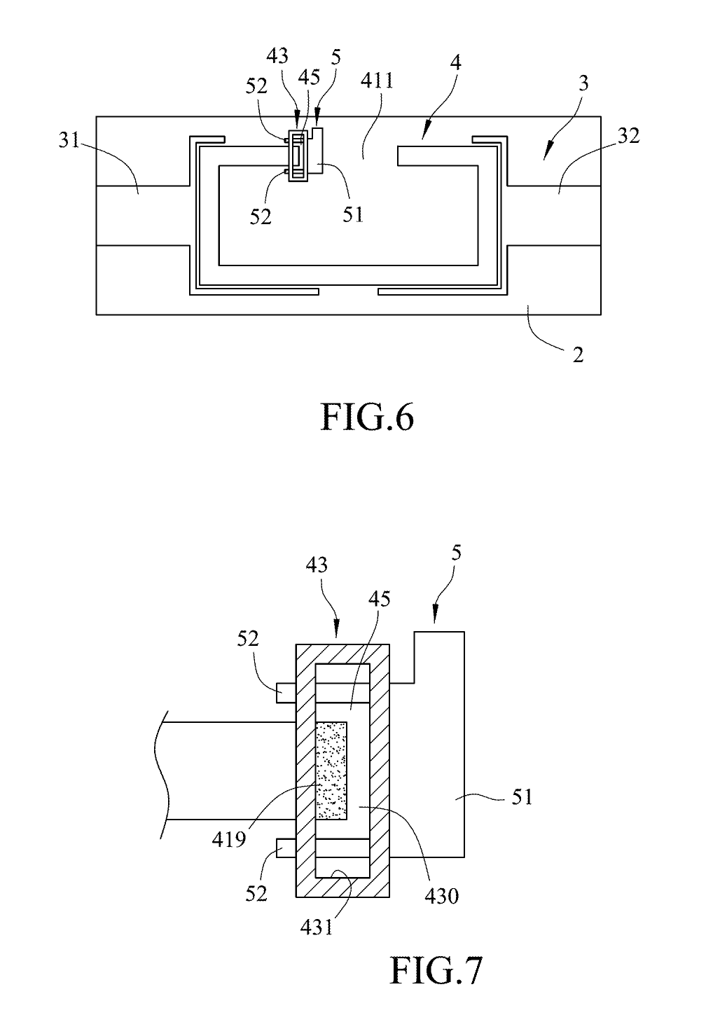

Referring to FIGS. 6 and 7, a second embodiment of the biosensor is illustrated. The second embodiment is similar to the first embodiment, but is different in that the biosensor further includes a limiter 43, and that the collection actuator 5 is disposed adjacent to one end of the microstrip resonator 4 beside the split 411 and cooperates with the microstrip resonator 4 to define a gap 45 therebetween. In practice, a size of the gap 45 is substantially in a range between 100 nanometers and 5 millimeters. The collecting portion 419 is formed at the end of the microstrip resonator 4, and the collection actuator 5 substantially surrounds the end of the microstrip resonator 4. The collection actuator 5 includes a main portion 51 and two actuator portions 52 extending from opposite ends of the main portion 51 along opposite sides of the microstrip resonator 4. Each of the two actuator portions 52 is narrower than the microstrip resonator 4. It is worth to note that the two actuator portions 52 may be provided with the ASR if the collection actuator 5 is made of a material allowing provision of the ASR thereon.

The limiter 43 is disposed at the microstrip resonator 4 and the collection actuator 5. The limiter 43 is formed with an inlet 431 and defines a receiving space 430 in spatial communication with the inlet 431 for accommodating the suspension. The collecting portion 419 is located in the receiving space 430, so the analyte, i.e., the antigen molecules 12, in the suspension may have more opportunity to bind with the ASR, i.e., the antibody molecules 11. The limiter 43 is made of a non-conductive material selected from the group consisting of silicone, polydimethylsiloxane (PDMS) and a combination thereof. It is worth noting that silicone has biocompatibility so the detection of the analyte would not be affected thereby.

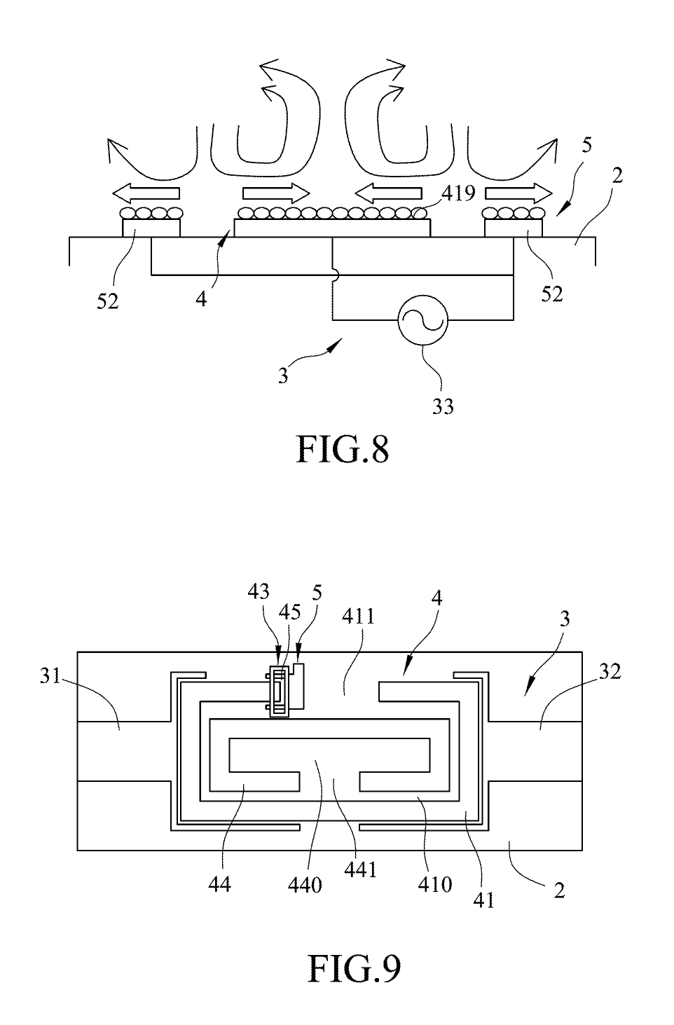

Referring to FIG. 8 in combination with FIGS. 6 and 7, the AC electric energy is applied to the microstrip resonator 4 and the two actuator portions 52 of the collection actuator 5 so as to induce AC electrokinetic stirring in the suspension for actuating the binding between the analyte and the ASR on the collecting portion 419. The second embodiment is not only capable of realizing the same detection as the first embodiment, but further enhances efficiency and precision of the detection. It should be noted that the receiving space 430 defined by the limiter 43 is not limited to what is illustrated in the Figures and description, and can be implemented according to a range of the collecting portion 419 as long as the analyte in the suspension can be confined in a certain space for facilitating detection.

Referring to FIG. 9, a third embodiment of the biosensor is illustrated. The third embodiment is similar to the second embodiment, but is different in that the microstrip resonator 4 is a split-ring resonator (SRR) including a pair of loops 41, 44 which are substantially concentric. Each of the loops 41, 44 is formed with a split 411/441 at a respective one of opposite sides of the pair of loops 41, 44. It should be noted that locations of the splits 411, 441 are not limited to what are illustrated in FIG. 9. The implementation of the microstrip resonator 4 in the third embodiment enhances further resonance so as to promote accuracy of detection. It is worth to note that the AC electric energy may also be applied to the loops 41, 44 so as to induce AC electrokinetic stirring in the suspension for actuating the binding between the analyte and the ASR. In other words, the loop 44 may play a role as the collection actuator 5 depending on different needs in design.

In summary, the biosensor of this disclosure integrates applications of the AC electrokinetic stirring and microstrip resonance. The AC electrokinetic stirring shortens the reaction time of binding between the analyte in the suspension and the ASR on the biosensor. Observing the shift in the resonant frequency of the microstrip resonator 4 through the network analyzer enables the analyte to be quantified.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A biosensor configured for detection of an analyte in a suspension when used in combination with a network analyzer, said biosensor comprising:
a substrate;
a microstrip resonator disposed on said substrate, and including a collecting portion that is provided with an analyte-specific reagent (ASR) thereon for binding the analyte in the suspension through affinity between said ASR and the analyte;
a collection actuator disposed on said substrate, and disposed adjacent to and spaced apart from said microstrip resonator, said collection actuator and said microstrip resonator being applied with alternating current (AC) electric energy so as to induce AC electrokinetic stirring in the suspension for actuating the binding between said ASR and the analyte; and
a port unit disposed on said substrate, and configured to be electrically connected to the network analyzer, and to be electromagnetically coupled with said microstrip resonator such that said microstrip resonator resonates at a resonant frequency which varies based on the binding between said ASR and the analyte, so as to enable the analyte to be quantified according to variation in the resonant frequency observed through the network analyzer.

2. The biosensor as claimed in claim 1, wherein said microstrip resonator is a single split-ring resonator formed with a split.

3. The biosensor as claimed in claim 2, wherein:
said collection actuator is disposed adjacent to one end of said microstrip resonator beside said split and cooperates with said microstrip resonator to define a gap therebetween; and
said collecting portion is formed at said end of said microstrip resonator.

4. The biosensor as claimed in claim 3, wherein said collection actuator substantially surrounds said end of said microstrip resonator.

5. The biosensor as claimed in claim 3, wherein said collection actuator includes a main portion and two actuator portions extending from opposite ends of said main portion along opposite sides of said microstrip resonator, each of said two actuator portions being narrower than said microstrip resonator.

6. The biosensor as claimed in claim 3, wherein a size of said gap is substantially in a range between 100 nanometers and 5 millimeters.

7. The biosensor as claimed in claim 1, wherein said port unit includes a first port and a second port via which said port unit is electrically connected to the network analyzer.

8. The biosensor as claimed in claim 7, wherein said microstrip resonator is disposed between said first and second ports of said port unit.

9. The biosensor as claimed in claim 1, further comprising a limiter disposed at said microstrip resonator and said collection actuator, formed with an inlet and defining a receiving space in spatial communication with said inlet for accommodating the suspension, said collecting portion being located in said receiving space.

10. The biosensor as claimed in claim 9, wherein said limiter is made of a non-conductive material selected from the group consisting of silicone, polydimethylsiloxane (PDMS) and a combination thereof.

11. The biosensor as claimed in claim 1, wherein said microstrip resonator is a split-ring resonator (SRR) including a pair of loops that are substantially concentric, and each of said loops is formed with a split at a respective one of opposite sides of said pair of loops.

12. The biosensor as claimed in claim 1, wherein:
said substrate has a height substantially in a range between 0.1 millimeters and 30 millimeters, and a relative dielectric constant substantially in a range between 1 and 50;
a length of said microstrip resonator is substantially in a range between 1 millimeter and 50 centimeters; and
said biosensor has an effective dielectric constant substantially in a range between 1 and 10000 when an effective width of said microstrip resonator is smaller than the height of said substrate, and substantially in a range between 1 and 100 when the effective width of said microstrip resonator is greater than the height of said substrate.

13. The biosensor as claimed in claim 1, wherein said collection actuator is formed on a portion of said port unit adjacent to said microstrip resonator.

14. The biosensor as claimed in claim 1, wherein said microstrip resonator resonates at the resonant frequency substantially in a range between 0.3 gigahertz and 100 gigahertz.

15. The biosensor as claimed in claim 1, wherein said microstrip resonator is made of a conductive material selected from the group consisting of metal, carbon and a combination thereof.

16. The biosensor as claimed in claim 1, wherein a thickness of said microstrip resonator is substantially in a range between 1 nanometer and 100 micrometers.

17. The biosensor as claimed in claim 1, wherein the AC electric energy has a peak-to-peak voltage substantially in a range between 0.01 volts and 30 volts, and a frequency substantially in a range between 0.1 hertz and 100 megahertz.

18. The biosensor as claimed in claim 1, wherein said microstrip resonator is an open-end resonator.

19. The biosensor as claimed in claim 1, wherein said microstrip resonator is a stub resonator.
